(19) United States
(12) Patent Application Publication
(10) Pub. No.: US 2017/0199123 A1
(43) Pub. Date: Jul. 13, 2017

(54) DETECTION METHOD OF HEAVY METAL IONS AND SENSOR USING THE SAME

(71) Applicant: GWANGJU INSTITUTE OF SCIENCE AND TECHNOLOGY, Gwangju (KR)

(72) Inventors: Min-Gon KIM, Gwangju (KR); Jin-Ho PARK, Gwangju (KR); Ju-Young BYUN, Gwangju (KR); Sang-Youp YIM, Gwangju (KR)

(21) Appl. No.: 15/361,491

(22) Filed: Nov. 28, 2016

(30) Foreign Application Priority Data
Jan. 7, 2016 (KR) 10-2016-0001905

(51) Int. Cl. G01N 21/552; G01N 33/18

(52) U.S. Cl. CPC G01N 21/554; G01N 33/1813; G01N 2201/12

(57) ABSTRACT

The present invention relates to a heavy metal ion detection method and sensor which can ascertain the presence of heavy metal ions based on a precipitation reaction with a reaction solution without using a receptor and is reusable after being washed. The heavy metal ion detection method includes: mixing a sample containing heavy metal ions with a reaction solution capable of forming a precipitate through reaction with the heavy metal ions on a dielectric substrate with metal nanoparticles attached thereto to form a precipitate; and measuring change in LSPR absorption wavelength of the metal nanoparticles before and after mixing to calculate a concentration of the heavy metal ions.

### CROSS REFERENCE TO RELATED APPLICATION

This application claims the benefit of Korean Patent Application No. 10-2016-0001905, filed on Jan. 7, 2016, entitled “DETECTION METHOD OF HEAVY METAL IONS AND SENSOR USING THE SAME”, which is hereby incorporated by reference in its entirety into this application.

### BACKGROUND

1. Technical Field

The present invention relates to a heavy metal ion detection method and sensor, and, more particularly, to a heavy metal ion detection method and sensor which can ascertain the presence of heavy metal ions based on a precipitation reaction with a reaction solution without using a receptor and is reusable after being washed.

2. Description of the Related Art

Heavy metals are raising concerns among ordinary persons knowing harmfulness thereof, as well as in scientific communities, particularly, among chemists, biologists, and environmental scientists. Particularly, mercury is a very toxic pollutant produced by natural phenomena or human activities.

Some microorganisms produce methyl mercury from another type of mercury. Methyl mercury is a neurotoxin and can damage the central nervous and endocrine system, causing various cognitive and behavioral disorders. This is because the methyl mercury is readily absorbed into the human body through the skin, respiratory organs, digestive organs, and the like. In addition, mercury can be propagated through various media including air, food, and water. This is alarming in that mercury propagated in this way can be accumulated through the food chain without being eliminated from an ecological system.

In order to solve problems due to exposure to mercury, there have been developed various methods for monitoring mercury ions (Hg²⁺) in biological and environmental samples. However, typical analysis methods including atomic absorption/emission spectrometry, cold vapor atomic fluorescence spectrometry, and inductively coupled plasma mass spectrometry are very costly and require well-trained operators.

In addition, such heavy metal detection methods require expensive equipment and are time consuming and difficult to conduct. In order to overcome these problems, there have been proposed methods of detecting mercury ions (Hg²⁺) in an aqueous solution using DNA-based enzymes, oligonucleotide, fluorescent quenchers, and the like. However, such methods require a detection receptor having a complex composition.

Therefore, there is a need for a heavy metal ion detection method or apparatus which can provide in-situ ascertainment of the presence of heavy metal ions and detection of the concentration of the heavy metal ions in a simple manner using a sample taken from a river, a lake, and the like, contaminable by heavy metals.

The information disclosed in this section is provided only for better understanding of the background of the invention, and should not be taken as an acknowledgment or any form of suggestion that this information forms prior art that would already be known to those skilled in the art.

### BRIEF SUMMARY

The present invention have been conceived to solve such a problem in the art and it is an aspect of the present invention to provide a heavy metal ion detection method and sensor which can detect heavy metal ions in water in a simple manner, allow quantitative analysis of the concentration of the heavy metal ions in a wider range, and exhibit improved detection sensitivity.

In accordance with one aspect of the present invention, a heavy metal ion detection method includes: mixing a sample containing heavy metal ions with a reaction solution capable of forming a precipitate through reaction with the heavy metal ions on a dielectric substrate with metal nanoparticles attached thereto to form a precipitate; and measuring change in a localized surface plasmon resonance (LSPR) absorption wavelength of the metal nanoparticles before and after mixing to calculate a concentration of the heavy metal ions.

The metal nanoparticles may be gold, silver, or platinum nanoparticles.

The heavy metal ions may be mercury ions (Hg²⁺).

The reaction solution may include citrate.

In accordance with another aspect of the present invention, a heavy metal ion detection sensor includes: a dielectric substrate; metal nanoparticles formed on the substrate; and a layer of a reaction solution applied to the metal nanoparticles to cover the metal nanoparticles and capable of forming a precipitate with heavy metal ions.

The metal nanoparticles may be gold, silver, or platinum nanoparticles.

The heavy metal ions may be mercury ions.

The reaction solution may include citrate.

Advantageous effects of the mercury ion detection method and sensor according to the present invention are as follows:

First, it is possible to detect heavy metal ions on site in a simple manner.

Second, it is possible to precisely detect mercury ions (Hg²⁺) in water containing other heavy metal ions due to selective precipitation of the citrate solution with mercury.

Third, it is possible to measure the concentration of mercury ions within a wider range from 1 mM (a relatively high concentration level) to 1 nM (meeting the acceptable concentration level).

Fourth, it is possible to reuse a mercury ion detection sensor by washing the sensor with distilled water, thereby allowing reduction in detection costs.

Fifth, it is unnecessary to additionally immobilize or prepare a specific sensing material on the sensor, whereby time and costs required for manufacture of the sensor can be reduced.

### BRIEF DESCRIPTION OF THE DRAWINGS

The above and other aspects, features, and advantages of the present invention will become apparent from the detailed description of the following embodiments in conjunction with the accompanying drawings, in which:

FIG. 1 is an image showing results of reacting various heavy metals with various reaction solutions;

FIG. 2 is a view illustrating a mercury ion (Hg²⁺) detection method according to the present invention;

FIG. 3 shows images (a) of mixtures of various heavy metal ions and citrate and a graph showing LSPR redshift measured when heavy metal ions are added to gold nanorods with a citrate solution applied thereto;

FIG. 4 shows Raman spectra measured on (a) mercury nitrate, (b) citrate powder, and (c) a mercury ion-citrate precipitate (exposed at a wavelength of 758 nm for 5 seconds);

FIG. 5 shows SEM images and LSPR spectra of (a) bare GNRs, (b) citrate/GNRs, (c) Hg²⁺/GNRs (d) Hg²⁺-citrate/GNRs, and (e) Hg²⁺/NaBH₄/GNRs;

FIG. 6 is a graph showing changes in adsorption spectrum with varying mercury ion (Hg²⁺) concentrations;

FIG. 7 is a graph showing detectable mercury ion (Hg²⁺) concentrations; and

FIG. 8 is a graph showing results of reusing a heavy metal ion detection sensor 8 times after washing the sensor with ultrapure water (DI water).

### DETAILED DESCRIPTION

The terminology used herein is for the purpose of describing particular embodiments and is not intended to be limiting. As used herein, the singular forms, “a,” “an,” and “the” are intended to include the plural forms as well, unless context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Unless otherwise defined herein, all terms including technical or scientific terms used herein have the same meanings as commonly understood by those skilled in the art to which the present invention pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, embodiments of the present invention will be described with reference to the accompanying drawings.

Typical methods of detecting mercury in water based on localized surface plasmon resonance (LSPR) may be divided into methods based on color change induced by specifically colored nanoparticles and methods based on amalgamation. Such methods have two problems; low detection sensitivity and narrow detection range, and poor reusability. Although a solution for any one of the above problems has been frequently reported, a solution for both of the above problems has not been reported yet.

The present inventors have completed the present invention based on the fact that the LSPR absorption wavelength can be shifted using a citrate solution having high reaction selectivity to mercury.

FIG. 1 is an image showing results of reacting various reaction solutions with various heavy metal ions. As shown in FIG. 1, heavy metal ions form a precipitate with a reaction solution. Thus, it is possible to ascertain the presence of a specific heavy metal using a reaction solution which forms a precipitate with the heavy metal.

Particularly, citrate forms a precipitate with mercury. Further, since citrate dose not react with heavy metal ions other than mercury ions, a citrate solution can be used to only detect mercury ions, regardless of other heavy metals.

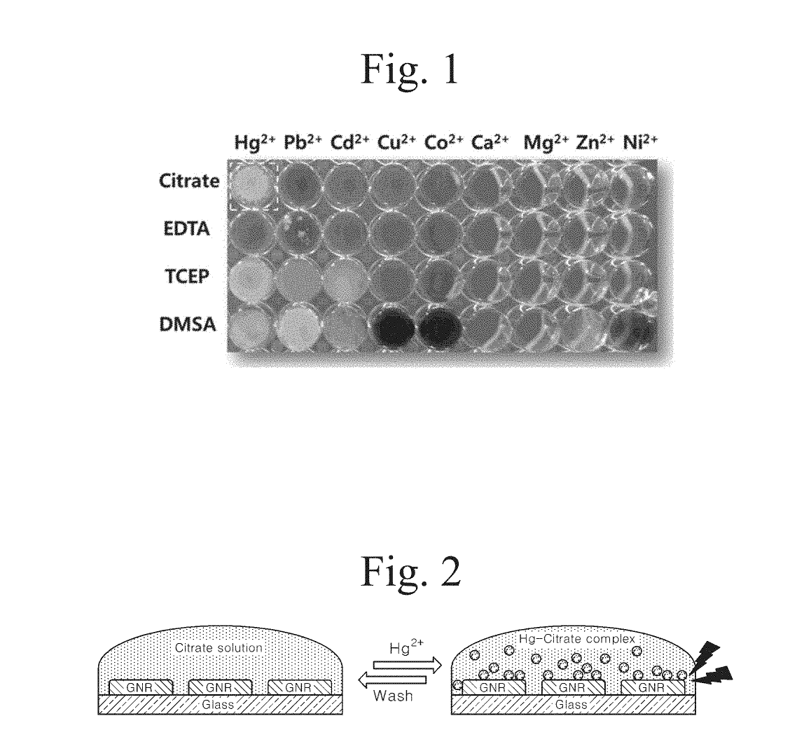

FIG. 2 is a view illustrating a heavy metal ion detection method according to one embodiment of the present invention. Referring to FIG. 2, in order to detect mercury ions, a substrate is prepared using glass as a dielectric, and gold nanorods (GNRs), as metal nanoparticles, are formed on the substrate, followed by application of a reaction solution containing citrate capable of forming a precipitate with mercury. A sample containing mercury ions is introduced into the applied citrate solution, thereby forming a precipitate. When the precipitate is attached to the gold nanorods, followed by measurement of LSPR absorbance of the gold nanorods, shift of absorption wavelength can be observed. A degree of shift of absorption wavelength is linearly changed with the amount of the precipitate. Thus, by measuring a variance in absorption wavelength, it is possible to calculate concentration of mercury ions (Hg²⁺) in a sample.

Although the present invention has been described using an example in which heavy metal ions to be detected are mercury ions, it should be understood that detectable heavy metal ions are not limited to mercury ions (Hg²⁺). In other words, using a reaction solution which contains various reactive groups capable of forming a precipitate with heavy metal ions and thus can be chelated with the heavy metal ions to form a precipitate, it is possible to detect various heavy metal ions.

The present invention is based on the fact that shift of absorption wavelength is observed due to a precipitate containing heavy metal ions upon measurement of LSPR absorbance of metal nanoparticles. Thus, it is possible to calculate concentration of any heavy metal ions by measuring a degree of shift of absorption wavelength so long as the heavy metal ions can form a precipitate with a suitable reaction solution, causing shift of absorption wavelength.

Next, the present invention will be described in more detail with reference to Experimental Examples.

### 1. Preparation of Gold Nanorods

Gold nanorods according to the present invention were prepared by a seed growth method. Put briefly, 3.75 ml of 0.1 M CTAB, 1.4 ml pf 0.01 M HAuCl₄, and 0.3 ml of 0.01 M NaBH₄ were mixed to prepare a seed solution. In addition, 33.04 ml of 0.1 M CTAB, 1.4 ml of 0.01 M HAuCl₄, 0.21 ml of 0.01 M AgNO₃, and 0.21 ml of 0.1 M ascorbic acid were mixed to prepare a growth solution. 0.14 ml of the seed solution is added to the growth solution, followed by reaction for 3 hours, thereby obtaining a gold nanorod solution. A UV/vis absorption spectrum of the gold nanorod solution was measured using a 96 well plate reader (Infinite M200pro, TECAN Group, Ltd., Switzerland).

### 2. Fabrication of Gold Nanorod Substrate

A substrate with gold nanorods attached thereto according to the present invention was fabricated in the following manner. A glass slide (10 mm×25 mm×0.7 mm) was washed in a piranha solution (H₂SO₄:H₂O₂=3:1) at 65° C. for 30 minutes. The glass slide was washed using ultrapure water (DI water) and ethanol and then immersed in a 2% ethanol APTMS solution at room temperature for 1 hour, thereby forming amine on a surface of the glass slide. Then, the glass slide was immersed in 1 M succinic anhydride at 37° C. for 12 hours, followed by washing the glass slide using ultrapure water (DI water). After 100 mM cysteamine hydrochloride was added to the ultrapure water in which the glass slide was immersed, the glass slide was treated using 100 mM EDC and 25 mM NHS to facilitate bonding between carboxylic groups of the succinic anhydride and amine groups of the cysteamine on the glass slide. After introduction of thiol to the glass slide, the glass slide was exposed to the gold nanorod solution for 20 hours more. Before the glass slide was passivated with gold nanorods, CTAB of the gold nanorods was removed.

### 3. Measurement Process

A 5 mM sodium citrate solution was applied to the substrate with gold nanorods formed thereon, followed by exposure to mercury ions having various concentrations. After about 15 minutes, optical absorbance of the substrate was measured at 6 places at intervals of 1 mm in a scanning mode. An absorption spectrum was measured at 25° C. at a wavelength of 700 nm to 850 nm, followed by calculating a variance in absorption wavelength using the origin program.

### 4. LSPR Redshift Caused by Precipitate Formed Through Reaction Between Mercury Ion (Hg²⁺) and Citrate

LSPR of metal nanoparticles is sensitively changed by a material attached to surfaces of the metal nanoparticles. Bonds between the material and the metal nanoparticles cause change in partial refractive index, resulting in shift of LSPR wavelengths. Based on such specific optical characteristics, it is possible to detect target molecules in a simple and sensitive manner.

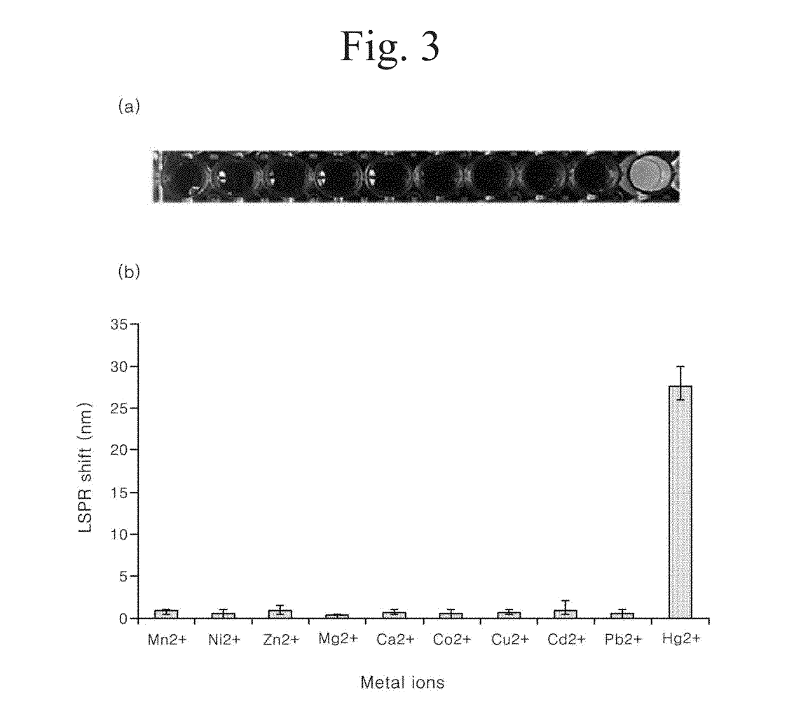

In order to ascertain selective reactivity between citrate and mercury ions (Hg²⁺), solutions containing various heavy metal ions were reacted with a citrate solution. Here, concentration of citrate in the citrate solution was 5 mM, and each of the solutions containing heavy metal ions had a heavy metal ion concentration of 1 mM. FIG. 3 shows images (a) of mixtures of various heavy metal ions and the citrate solution and a graph showing an LSPR redshift measured when the heavy metal ions were added to gold nanorods with the citrate solution applied thereto. As shown in FIG. 3, it could be seen that mercury ions formed a precipitate with the citrate solution, whereas the other heavy metal ions did not react with the citrate solution. In addition, it was ascertained that the precipitate changed the index of refraction on surfaces of the gold nanorods, causing an LSPR redshift of 27.67 nm. Thus, based on such an LSPR redshift, it is possible to provide a mercury ion detection sensor which can detect mercury ions without any interruption by other heavy metal ions, thereby eliminating a need for a specific receptor.

### 5. Raman Analysis of Mercury Ion-Citrate Complex Precipitate

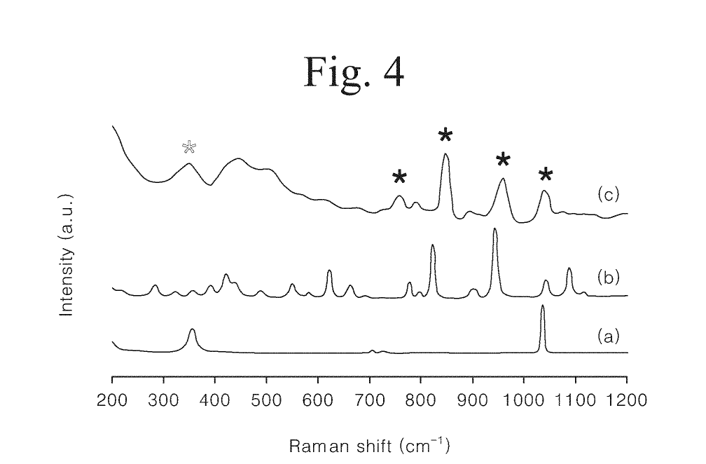

Bonds between mercury ions (Hg²⁺) and carboxylic groups of citrate were analyzed by Raman spectroscopy. FIG. 4 shows Raman spectra measured on (a) mercury nitrate, (b) citrate powder, and (c) a mercury ion-citrate complex precipitate (exposed at a wavelength of 758 nm for 5 seconds). As shown in FIG. 4, the Raman spectrum of the citrate has peak values of 823 cm⁻¹ and 944 cm⁻¹, which are attributed to three stretched C-C bonds. In contrast, the peaks of the Raman spectrum of the mercury ion-citrate complex precipitate are red-shifted to 847 cm⁻¹ and 959 cm⁻¹, respectively. This is due to the fact that carboxylic groups of the citrate are chelated with mercury ions (Hg²⁺).

### 6. Determination of Amalgamation

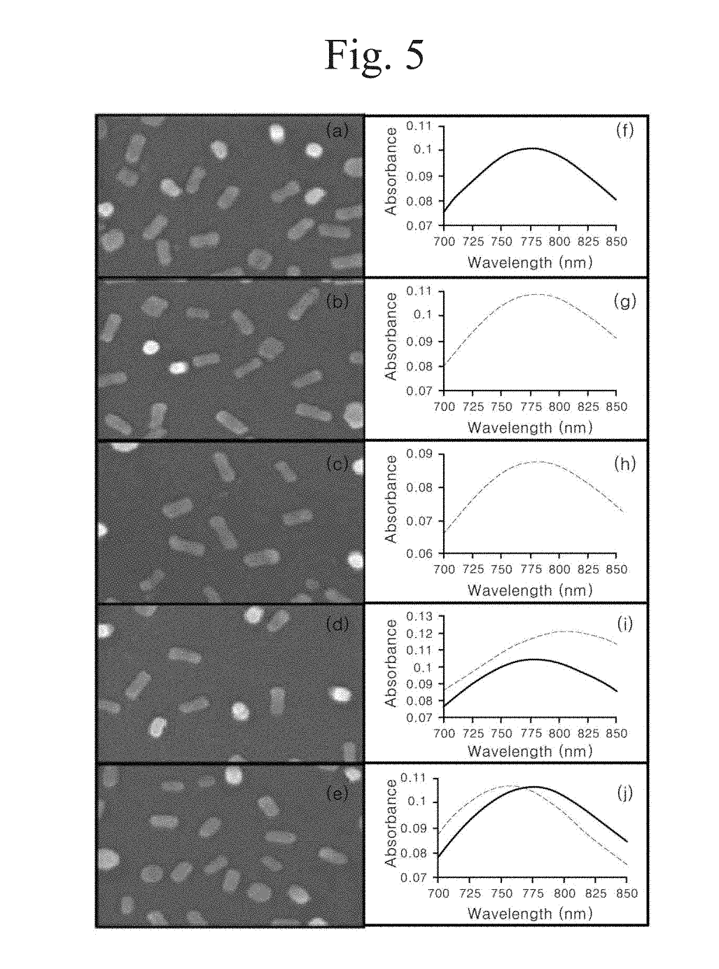

In a typical method of detecting mercury ions (Hg²⁺), gold particles form an amalgam with mercury ions (Hg²⁺). In the present invention, a precipitate is formed by coordination bonds between mercury ions (Hg²⁺) and citrate, causing LSPR redshift. As such, the heavy metal ion detection sensor according to the present invention does not cause amalgamation and thus can be reused after being washed. FIG. 5 shows SEM images and LSPR spectra of (a) bare GNRs, (b) citrate/GNRs, (c) Hg²⁺/GNRs (d) Hg²⁺-citrate/GNRs, and (e) Hg²⁺/NaBH₄/GNRs. As shown in FIG. 5, it can be seen that, in the case of (e) undergoing amalgamation, the length of GNRs is reduced, whereas, in the cases other than (e), the length of GNRs remains unchanged since amalgamation does not occur.

### 7. Quantitative Analysis on Mercury Ion (Hg²⁺)

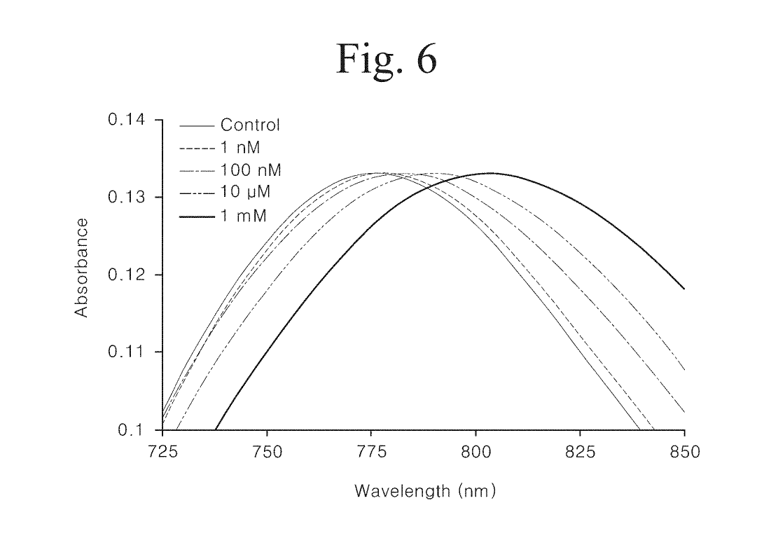

In order to verify quantitative analysis capability of the mercury detection method according to the present invention, solutions having various mercury ion (Hg²⁺) concentrations were added to a gold nanorod substrate with citrate applied thereto. FIG. 6 is a graph showing changes in adsorption spectrum with varying mercury ion (Hg²⁺) concentrations. As shown in FIG. 6, it can be seen that, as the mercury ion (Hg²⁺) concentration is increased, the LSPR absorption wavelength is red-shifted. From the results, it can be seen that the content of mercury ions can be measured within the range of 1 nM to 1 mM, based on a value of redshift of the LSPR absorption wavelength.

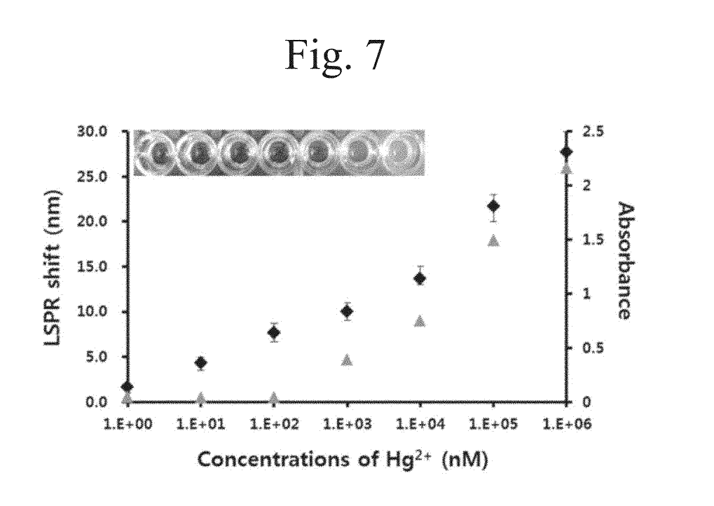

FIG. 7 is a graph showing detectable mercury ion (Hg²⁺) concentrations. As shown in FIG. 7, it can be seen that, when the redshift of the LSPR absorption spectrum is measured by the mercury detection method according to the present invention, it is possible to calculate the mercury ion concentration in nMs, whereas, when the absorption spectrum at 300 nm caused by a mercury ion (Hg²⁺)-citrate complex precipitate is measured, the mercury ion concentration can be calculated in μMs or more.

### 8. Evaluation of Reusability of Mercury Ion (Hg²⁺) Detection Sensor

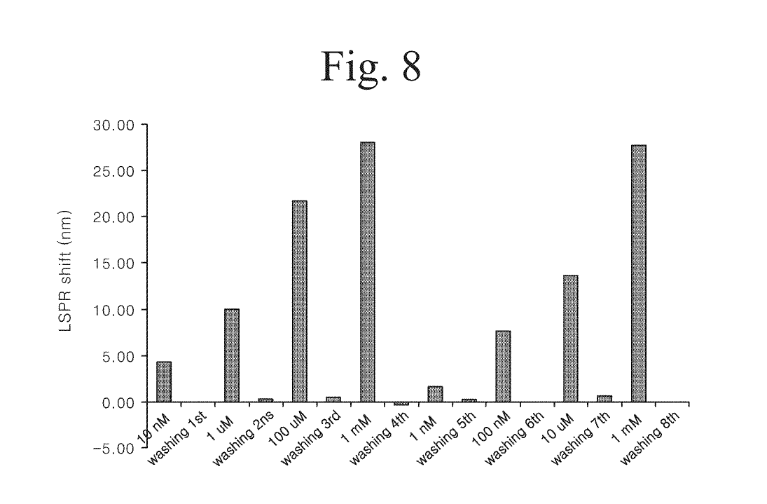

Advantageously, the mercury ion detection sensor according to the present invention is reusable after a detection reaction. In other words, a mercury ion (Hg²⁺)-citrate complex precipitate attached to gold nanorods can be removed from the gold nanorods through a simple washing process. FIG. 8 is a graph showing results of reusing the mercury ion detection sensor 8 times after washing with ultrapure water (DI water). As shown in FIG. 8, it could be seen that, when solution samples containing mercury ions (Hg²⁺) having various concentrations were used in a detection test, followed by a retest subsequent to washing with ultrapure water (DI water), the mercury ion detection sensor provided high reproducibility.

Although some embodiments have been described herein, it should be understood by those skilled in the art that these embodiments are given by way of illustration only, and that various modifications, variations and alterations can be made without departing from the spirit and scope of the invention. Therefore, the embodiments and the accompanying drawings should not be construed as limiting the spirit of the present invention, but should be construed as illustrating the spirit of the present invention. The scope of the invention should be interpreted according to the following appended claims as covering all modifications or variations derived from the appended claims and equivalents thereof.

What is claimed is:

1. A heavy metal ion detection method, comprising:
mixing a sample containing heavy metal ions with a reaction solution capable of forming a precipitate through reaction with the heavy metal ions on a dielectric substrate with metal nanoparticles attached thereto to form a precipitate; and
measuring change in a localized surface plasmon resonance (LSPR) absorption wavelength of the metal nanoparticles before and after mixing to calculate a concentration of the heavy metal ions.

2. The heavy metal ion detection method according to claim 1, wherein the metal nanoparticles are gold, silver, or platinum nanoparticles.

3. The heavy metal ion detection method according to claim 1, wherein the heavy metal ions are mercury ions (Hg²⁺).

4. The heavy metal ion detection method according to claim 1, wherein the reaction solution comprises citrate.

5. A heavy metal ion detection method, comprising:
preparing a substrate by depositing gold nanoparticles on a dielectric;
measuring an LSPR absorption wavelength of the gold nanoparticles;
applying a reaction solution containing citrate to the substrate;
adding a sample containing mercury ions (Hg²⁺) to the reaction solution to form a precipitate on the gold nanoparticles;
measuring an LSPR absorption wavelength of the gold nanoparticles with the precipitate attached thereto; and
calculating concentration of mercury ions (Hg²⁺) by measuring change in the LSPR absorption wavelength before and after attachment of the precipitate.

6. A heavy metal ion detection sensor, comprising:
a dielectric substrate;
metal nanoparticles formed on the substrate; and
a layer of a reaction solution applied to the metal nanoparticles to cover the metal nanoparticles and capable of forming a precipitate with heavy metal ions.

7. The heavy metal ion detection sensor according to claim 6, wherein the metal nanoparticles are gold, silver, or platinum nanoparticles.

8. The heavy metal ion detection sensor according to claim 6, wherein the heavy metal ions are mercury ions.

9. The heavy metal ion detection sensor according to claim 6, wherein the reaction solution comprises citrate.
